(19) United States
(12) Patent Application Publication
(10) Pub. No.: US 2017/0136044 A1
(43) Pub. Date: May 18, 2017

(54) METHOD OF TREATING PRE-CANCEROUS LESIONS WITH GLYPHOSATE, AND COMPOSITIONS THEREOF

(71) Applicant: CALVIN NEYMEYER, CLINTON, IA (US)

(72) Inventor: CALVIN NEYMEYER, CLINTON, IA (US)

(21) Appl. No.: 14/939,968

(22) Filed: Nov. 12, 2015

(51) Int. Cl. A61K 31/662

(52) U.S. Cl. CPC A61K 31/662

(57) ABSTRACT

A method for treating a pre-cancerous skin lesion on a human subject with glyphosate is described, along with compositions for said treatment.

### CROSS-REFERENCE TO RELATED APPLICATIONS

This application claims the benefit of U.S. Provisional Application No. 62/078/103, filed Nov. 1, 2014, and entitled “METHOD OF TREATING PRE-CANCEROUS LESION”, said provisional application hereby incorporated by reference in its entirety.

### FIELD OF THE INVENTION

This invention is directed to the use of glyphosate compositions to treat pm-cancerous skin lesions.

### BACKGROUND OF THE INVENTION

Skin cancer is a common form of cancer, with thousands of people diagnosed and dying from it every year Pre-cancerous lesions such as actinic keratosis may precede the formation and diagnosis of cancer of the skin, and cause discomfort and other problems.

Glyphosate is an effective but controversial herbicide, with environmentalist and other groups concerned in part with mild to moderate systemic and dermal toxicity reported from contact with glyphosate-containing compositions. Amerio et al, “Skin Toxicity from Glyphosate-Surfactant Formulation” J. Toxicology CLINICAL TOXICOLOGY 42(3)317-19 (2004).

A safe, efficacious treatment for pre-cancerous lesions would be beneficial.

### BRIEF SUMMARY OF THE INVENTION

The present invention is directed to the use of glyphosate-containing compositions in the treatment of pre-cancerous skin lesions. A glyphosate composition is administered by directly, topically applying the composition to a lesion. The lesion disappears and appears to be fully cured within weeks or even days, such that the skin regains the appearance of adjacent, healthy skin. The present invention is also directed to a composition comprising glyphosate for the treatment of precancerous skin lesions; and to the use of a composition comprising glyphosate, for the preparation of a medicament for the treatment of a pre-cancerous skin lesion,

### DETAILED DESCRIPTION OF THE INVENTION

The present invention is directed to a method for treating a pre-cancerous lesion with glyphosate, comprising the step of topically applying the glyphosate to the lesion in a therapeutically effective amount.

The present invention is also directed to a composition comprising glyphosate for instance as discussed above, for the treatment of a pre-cancerous skin lesion. Also, the present invention is directed to the use of a composition comprising glyphosate of the present invention, for the preparation of a medicament for the treatment of a precancerous skin lesion.

For the purposes of the present invention, “skin cancer” refers to any type of cancer of the skin including for instance melanoma basal cell cancer, or squamous cell cancer. A “pre-cancerous lesion” refers to a change in the skin that is not yet cancerous but may become so including for instance an actinic keratosis. Preferably, a pre-cancerous lesion is a small reddened raised area that may have a small scab, as described in the Examples.

The term “topical” and the like refers to the external surface of the skin. A composition having glyphosate is administered according to the present invention through topical application of the composition to a pre-cancerous lesion; placing the composition in direct contact with the lesion. Topical application can include a variety of known methods, including by drop, by application via finger, or for instance a cotton pad. Preferably, the composition is applied to an abraded lesion with gentle rubbing.

The terms “treat”, “treating”, and “treatment”, and the like, refer to ameliorating (improving or making less severe) and/or preferably to curing a pre-cancerous lesion. Said treatment may occur in a human or non-human subject having a pre-cancerous lesion. Preferably, a pre-cancerous lesion treated according to the present invention is fully cured, and the skin healed. Treatment according to the present invention may be achieved by applying a composition of the present invention one or more times to the pre-cancerous lesion. Preferably, the composition is applied at least two, and more preferably at least three, times during a course of treatment. Also preferably, the composition is applied fewer than 20 times, more preferably fewer than 10 times, more preferably fewer than 5 times, and most preferably 3 times, 2 times, or 1 time during the treatment of a given lesion or cancer, Multiple applications are preferably spaced 3 days to 14 days apart. The composition of the present invention may be applied for instance 3 times, for instance one time every three days; or one time alone; or two times, for instance one week apart. Other treatment schedules are also contemplated with regard to this invention. While the preferred treatment is a full cure, a composition of this invention may be applied for maintenance or preventative purposes. Also, during a treatment according to the present invention, compositions having a higher concentration of glyphosate may be used to provide a loading dose and a composition having a lower glyphosate concentration (preferably approximately one-half of the composition used for the loading dose) for remaining applications.

The phrase “therapeutically effective amount” refers to an amount of glyphosate ((2-(phosphonomethylamino) acetic acid) or its salts) sufficient to treat (preferably cure) a pre-cancerous lesion in a subject. Preferably a single application of glyphosate to a pre-cancerous lesion will include about 1 mg to about 400 mg glyphosate; more preferably, about 4 mg to about 100 mg; more preferably, about 3 mg to about 90 mg; more preferably, about 4 mg to about 75 mg; more preferably, about 5 mg to about 90 mg; most preferably, about 5 to about 30 mg, Amounts applied to a pre-cancerous lesion may be for instance 1, 2, 3, 4, 5, 6, 7, 8, 9, 10, 1120, 21-30, 31-40, 41-50, 51-60, 62-70, 71-60, 81-90, 91400, 101-110, 111-120, 121-130, 131-140, 141-150, 151-160, 161470, 171-180, 181-190, 191-200, 201-210, 211420, 221-230, 231-240, 241-250, 251-260, 261470, 271-280, 281-290, 291-300, 301-310, 311-320, 321-330, 331-340, 341-350, 351-360, 361-370, 371-380, 381-390, or 391-400 mg in a single application. The glyphosate may be present in an amount of about 1% to about 99% of the composition. The glyphosate may comprise for instance 1-5% glyphosate; 6-10% glyphosate; 11-15% glyphosate; 16-20% glyphosate; 21-25% glyphosate; 26-30% glyphosate; 31-35% glyphosate; 36-40% glyphosate; 41-45% glyphosate; 46-50% glyphosate; 51-55% glyphosate; 56-60% glyphosate; 61-65% glyphosate; 66-70% glyphosate; 71-75% glyphosate; 76-80% glyphosate; 81-85% glyphosate; 86-90% glyphosate; 91-95% glyphosate; or 96-99% glyphosate; by weight, of the total composition. The glyphosate may also be present in amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43.44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% by weight of the total composition. A range including the above amounts is also contemplated in the context of the present invention.

A composition of the present invention may include glyphosate alone, or with one or more active ingredients, preferably also useful in treating pre-cancerous lesions, and/or one or more inactive ingredients, for instance to provide effective delivery of the glyphosate. Such inactive ingredients include for instance water and minor formulating ingredients, as well as other compounds suitable for topical application. Preferably, the glyphosate bin the form of an isopropylamine salt. The composition may be a liquid solution, gal, cream, ointment, or other form suitable for topical application to the skin. Preferably, the composition is in aqueous solution, more preferably the composition has an acidic pH, even more preferably a pH in the range of about 15 to about 6, and more preferably, a pH in the range of 4-5.

The composition employed in the following; examples are products identified in EPA Reg No. 71995-29, (Roundup Weed & Grass Killer Concentrated Plus) EPA Reg No. 71995-27 and/or EPA Reg No. 71995-27-239 (Ortho Bask Solutions Weed & Grass Killer Ready-to-Use, spray bottle, 1.92% glyphosate (“Ortho composition”)). The MOS for these products are included herewith and incorporated by reference as needed to aid in describing compositions that may be used in the present invention.

The following examples are intended to exemplify the present invention, but not to be limiting. The inspiration for the invention came from the inventor mentally connecting the concept of the spread of plant spores to metastasis in the human body.

### EXAMPLE I

A pre-cancerous lesion (actinic keratosis) on an elderly man's face was treated with a glyphosate composition. The lesion was small, approximately 1 mm in size and located 20 mm to the left of the lower edge of the man's left nostril, The lesion persisted for approximately 8 months prior to treatment according to the present invention, resisting three attempts at treatment with alcohol and/or topical antibiotics, such that the lesion would redden slightly and trickle blood, but not heal.

A small scab covered the lesion prior to treatment according to the present invention. The scab, lesion, and area surrounding the lesion were gently cleansed with Dial liquid soap and the scab removed, to provide maximum exposure of the lesion to the glyphosate.

A small amount of Ortho composition (Ortho Basic Solutions Weed & Grass Killer Ready-to-Use composition, in an original spray bottle, having 1.92% glyphosate (isopropylamine salt)), was sprayed into the palm of the subject's hand and the cotton pad at the end of a Q-tip immersed in the Ortho composition. When the Q-tip was saturated with the Ortho composition, the saturated end of the Q-tip was gently but firmly pushed into the lesion. The Q-tip was twisted slightly to ensure maximum coverage and removed. This process was repeated. There was a slight stinging as the Ortho composition contacted the tissue. No other side effects were noted. The area was not washed again until the next morning. No other substances were applied to the lesion.

The lesion and surrounding area was observed daily, several times during the day, to follow the progression of the treated area. The redness began disappearing, and after four days, the skin was similar to surrounding tissue, although still with a slight difference. The area was slightly reddish, blotchy and there was a tiny crater where the lesion had been but appeared to be healing, no rawness, no scabbing, and the skin appeared normal The small lesion was treated and fully healed in 7 days, with just one application of the Ortho composition.

A second lesion (actinic keratosis) on the same male subject was on the left side of the subject's throat, about 60 mm directly below the subject's left ear. The lesion would not heal it would scab over for 3 to 4 days, and then the scab would leave, revealing the reddened unhealed area underneath. The lesion was covered with antibiotics several times, causing it to scab and eventually slough off, again revealing a reddened unhealed area. The lesion was sensitive when touched; care was taken during shaving to avoid pain and worsening of the lesion. This continued for 6-8 months.

The second lesion was treated with the Ortho composition. The lesion was thoroughly cleansed with Dial liquid soap to expose as much of the lesion as possible. Most of the lesion became exposed as the scab was gently removed. After cleaning, the lesion showed a raw, unhealed area with a crater-like appearance in the upper end of the unhealed area. The lesion was not rubbed vigorously, but was rubbed firmly, to expose as much lesion tissue as possible. The lesion was about 15 mm long and 8 mm wide, running diagonally upward toward the left ear, with the top of the lesion about 60 mm below the lobe of the left ear.

The Ortho composition was squirted into the subject's hand and end of Q-tip was immersed into the liquid, generally as discussed regarding the first lesion above. After gently squeezing the excess fluid from the Q-tip, the Ortho composition was topically applied to the lesion, so that the entire lesion was covered, with attempts to not extend application much over the borders of the lesion.

The next day the entire lesion was covered with a scab. The area was not further treated with the Ortho composition. The area was washed gently with Dial liquid soap and water; care was taken to not irritate the area when the subject shaved.

There was a slight stinging sensation when the Ortho composition was applied, similar to the above discussion of the first lesion. The sensation quickly passed and did not continue or repeat. Another similarity between treatment of the two lesions is that soon after application, the area felt better. There was a reduced sensation of discomfort. The lesion was observed daily for progress. The area was quiet, no discharge, and slightly less redness.

Four days later the scab appeared to begin to slough off on its lower edge. In the following days, this gradually moved upward, The area was less sensitive to pain but would still produce a pain sensation when rubbed or touched with a razor.

As the scab area sloughed off, the area under it was somewhat reddened but the tissue appeared healed. There was no rawness and the tissue appeared similar to the tissue adjoining it. This redness gradually lessened as time progressed.

Eighteen days later there was only a small round unhealed area, the size of a BB, that had not completely healed. It was in the area where there appeared to be a crater like formation at the upper end of the lesion. Five days later, the entire area was healed.

Only one application of the Ortho composition was made to the lesion.

### EXAMPLE II

The Ortho composition was directly and topically applied by gentle rubbing to a pre-cancerous skin lesion (actinic keratosis) on the face of a human male subject, just below his eye. Prior to treatment, the lesion was treated in the approximate manner described in Example I, it is estimated that approximately 5 mg of glyphosate was applied to the lesion. The lesion was approximately 8 mm wide and 3 mm long, mildly raised and somewhat painful to the touch, with a scaly, reddened appearance and a small area of broken skin with a small scab on it. The amount of composition applied was sufficient to cover and coat the lesion. The application was applied and, over the span of 2 days, the scab disappeared and redness and swelling lessened. No adverse effects were reported by the subject.

### EXAMPLE III

The Ortho composition of Example was applied to a different lesion (actinic keratosis) on the same subject, having similar characteristics to that described in Example II Application occurred 3 times; Day 0, Day 3, and Day 7. It is estimated that each application included approximately 5.0 mg of glyphosate. The scab disappeared and redness and swelling lessened and then disappeared over the span of the following 3 weeks. No adverse effects were reported by the subject.

### EXAMPLE IV

A composition (EPA Reg. N. 7105-29) having approximately 18% of isopropylamine salt of glyphosate in an aqueous solution, pH about 5, was directly applied by gentle rubbing to a pre-cancerous skin lesion (actinic keratosis) on the tip of the ear of a human male subject. The lesion was small (about 3 mm long and 2 mm wide) and scabbed, and was abraded prior to application of the composition to allow the composition direct access to the raw skin under the scab. A blood blister appeared over the lesion and was punctured and drained during treatment. The composition was reapplied 4 days after the initial application, with any scab again abraded. The lesion was completely healed within 10 days. Other than the formation of the blood blister, no adverse effects were reported by the subject.

1. A method of treating a pre-cancerous lesion of the skin, comprising the step of topically applying a composition having glyphosate to the lesion in a therapeutically effective amount.

2. The method of claim 1, wherein the glyphosate is in the form of an isopropylamine salt.

3. The method of claim 1, wherein the glyphosate comprises about 1% to about 99% by weight of the composition.

4. The method of claim 1, wherein the glyphosate comprises about 2 to about 20% by weight of the composition.

5. The method of claim 1, wherein the composition has a pH of about 3.5 to about 6.

6. The method of claim 1, wherein about 1 to about 100 mg of glyphosate is applied to a lesion in at least one application.

7. The method of claim 1, wherein about 50 to about 70 mg of glyphosate is applied to a lesion in at least one application.

8. The method of claim 1, wherein the composition is applied to a lesion at least twice over a period of from 3 to 7 days.

9. The method of claim 6, wherein the composition is applied to a lesion at least twice over a period of from 3 to 7 days.

10. The method of claim 1, wherein the composition is the subject of EPA Reg. No. 71995-29.

11. The method of claim 1, wherein the composition is the subject of EPA Reg. No. 71995-27.

12. The method of claim 10, wherein the composition is the subject of EPA Reg, No. 71995-27-239.
